(19) United States
(12) Patent Application Publication
(10) Pub. No.: US 2011/0071449 A1
(43) Pub. Date: Mar. 24, 2011

(54) Fulcrum Knee Brace

(75) Inventor: Duane A. Kuhler, Columbus, OH (US)

(73) Assignee: Duane A. Kuhler, Columbus, OH (US)

(21) Appl. No.: 12/586,212

(22) Filed: Sep. 18, 2009

(51) Int. Cl. A61F 5/00

(52) U.S. Cl. 602/16

(57) ABSTRACT

A brace for supporting a knee joint is disclosed which is specifically adapted to restrict lateral movement of the knee joint in addition to preventing hyperextension of the user's leg. The brace is formed by a rigid proximal member that fully covers the front and sides of the knee and extends partially over the tibia and a rigid distal member which overlaps the proximal member in the area of the knee joint while additionally extending over a portion of the upper leg of the user. The rigid members are attached together with a pair of hinge pins disposed on opposite sides of the knee with a primary pivot axis to allow bending of the knee joint. Attached to the inner surface of the proximal member is a knee pad which is used to provide a firm interface between the proximal member and the area of the knee joint. The knee brace further comprising at least two strap members attached to said shell members and extensible about the leg of the user.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The field of endeavor to which this invention pertains may be classified as subject matter comprising an appliance which is specifically adapted to have a point of flexure intermediate a plurality of substantially rigid splint elements to accommodate flexure of a natural joint. In the past, knee braces have not made allowances for protection of the knee cap when the user is kneeling upon a surface or when the user is exposed to an impact when standing or running. This invention serves to provide protection of the knee from impacts, such as that which would be encountered doing sports related activities. Additionally, this invention restricts sideways movement of the knee joint, as well as providing a means to prevent hyper-extension of the user's leg.

### BRIEF SUMMARY OF THE INVENTION

The advantage of the invention includes a means of supporting and protecting the user's knee from injury in three distinct ways.

The first type of protection created by the fulcrum knee brace is in the form of a hard shell which completely covers the front and sides of the user's knee joint thereby providing protection from impacts and abrasions. The brace comprises both an upper section and lower section which are attached together with connecting pins that are oppositely disposed on either side of the user's knee joint thereby allowing for the normal range of movement of the user's leg and knee joint and effectively providing continuous protection from impacts and abrasions to the frontal and side areas of the knee joint. Additionally, the brace comprises a means of being attached to the user's leg through the use of elasticized straps which are extensible around the back of user's leg and attached to the brace on either side.

The second type of protection is in the form of preventing hyperextension of the user's leg and knee joint by utilizing an integrated leverage point wherein as the user extends the leg to a straightened position, the upper brace section rotates upon the connecting pins and completely overlaps the lower brace section in the area of the knee joint, thus bringing the overlapping edge of the upper brace section into contact with the outer surface of the lower brace section, thereby limiting any further travel beyond that point and effectively preventing hyperextension of the user's leg.

The third type of protection is in the form of lateral support to the knee joint which is facilitated by the use of extended flanges which overlap on either side of the knee joint area and provide a stable bearing surface where the connecting pins are located. The upper and lower sections of the knee brace rotate around the axis of the oppositely disposed connecting pins thereby restricting the movement of the user's leg and knee to a single plane of travel, thus providing lateral stability to the knee joint, as well as the adjacent ligaments and connecting tissue of the user's leg.

### BRIEF DESCRIPTION OF THE SEVERAL VIEWS OF DRAWING

FIG. 1 is an exploded diagram of a knee brace consisting of a proximal section (1) with an overlapping distal section (2) and two connecting pins (3) located on opposite sides of the brace while sharing a common axis (4) so as to allow the said distal section to rotate around the said proximal section on the common axis being shared by the said opposing connecting pins.

FIG. 2 shows the assembled knee brace with the proximal section (1) being overlapped by the distal section (2) in the area of the knee as would be encountered when the user's leg is straightened. In this position, the overlapping edge (5) of the distal section makes contact with the outer surface (6) of the said proximal section. This point of contact prevents any further extension of the device, thereby preventing hyperextension of the user's leg and knee joint.

FIG. 3 depicts the proximal section (1) and distal section (2) of the knee brace attached together with the opposing connecting pins (3) located on the sides of the brace. The previously said distal section is shown rotated over the previously said proximal section around the axis of the previously said connecting pins, displaying how the brace completely covers the knee area as would be encountered when the knee is bent.

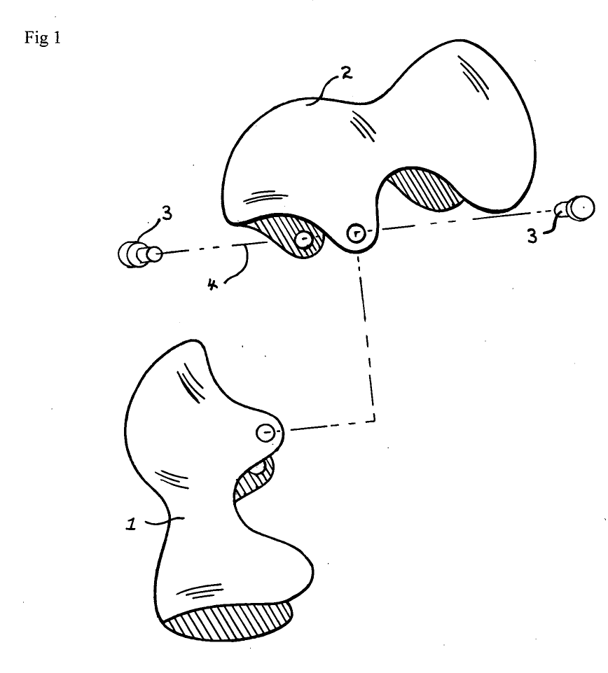

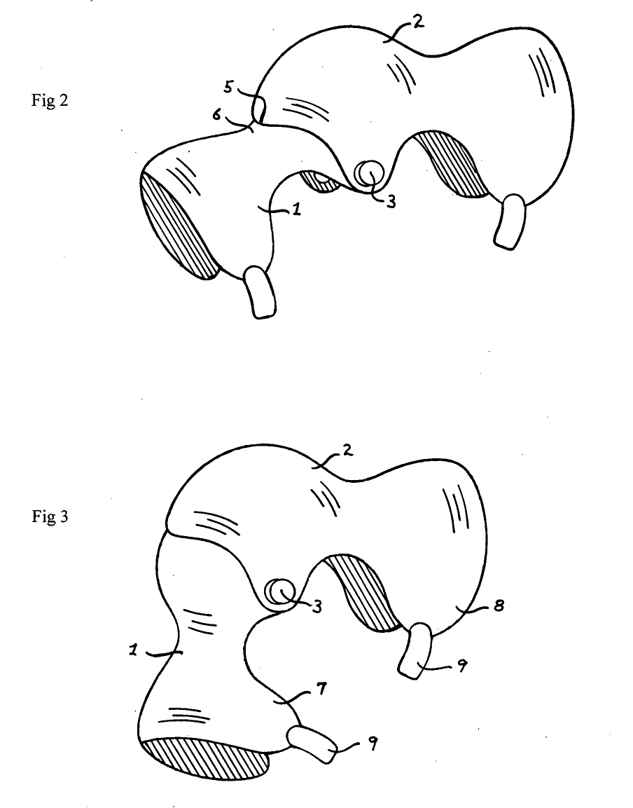

The extended flanges of the proximal member (7) conform to the upper calf area of the user's leg and stabilize the brace below the knee joint. The extended flanges of the distal member (8) conform to the lower thigh area of the user's leg and stabilize the brace above the knee joint.

The brace sections are secured in place by straps (9) wrapping around the back of the leg, and having a means of attachment so as to be easily removed and reapplied.

### DETAILED DESCRIPTION OF THE INVENTION

A brace for supporting a knee joint is disclosed which is specifically adapted to restrict lateral movement of the knee joint in addition to preventing hyperextension of the user's leg, while allowing normal movement of the knee joint when the brace is applied to the user's leg. The object of the invention is to provide protection to the ligaments and tendons of the knee joint by limiting the direction and range of movement of the user's leg while the user is engaged in sports related activities or other potentially hazardous activities. The knee brace consists of two separate sections, both comprised of a semi-rigid plastic material of composition such as that which would be commonly used in sports related equipment. The brace is formed by a rigid proximal member that fully covers the front and sides of the knee and extends partially over the tibia. Padding is attached to the inner surface to provide a firm interference between the rigid plastic and the user's leg. The brace further includes a rigid distal member which overlaps the proximal member in the area of the knee joint while additionally extending over a portion of the upper leg of the user. The rigid members are attached together with a pair of hinge pins disposed on opposite sides of the knee with a primary pivot axis to allow bending of the knee joint. The hinge pins are attached in a manner that will allow the upper distal section of the device to rotate freely around and overlap the lower proximal section in the area of the knee joint and closely following the normal range of motion of the knee. When the knee is bent the brace completely covers the knee area, continuing to provide protection to the knee cap. The brace further includes a means of preventing hyperextension of the user's leg and knee joint. This is accomplished as the user's leg is extended or straightened and the distal section rotates on the connecting pins, thereby overlapping the proximal section in the area of the knee joint until the rotation of the device brings the leading edge of the said distal section into contact with the outer surface of the proximal section of the brace, thereby preventing further rotation of the device and preventing hyperextension of the user's leg. Additionally, the brace includes a means of preventing lateral movement of the leg throughout the complete range of motion. This is accomplished with the use of extended flanges located on the sides of the proximal member that conform to the upper calf area of the user's leg and stabilizes the brace below the knee joint, and extended flanges of the distal member that conform to the lower thigh area of the user's leg and stabilizes the brace above the knee joint and a knee pad which is attached to the inner surface of the proximal member which is used to provide a firm interference between the proximal member and the area of the knee joint. The knee brace further comprises at least two strap members attached to the said shell members as a means of securing the device to the user's leg.

What is claimed is a knee brace comprising:

1.) A lower shell member that is proximal to the knee and an upper shell member that is distal to the knee, each formed from a rigid material, said proximal shell member fully covering the front and sides of the user's knee and extending partially over the tibia and said distal shell member overlapping the previously said proximal shell member in the area of the knee and extending partially over the upper leg area of the user, such that each said shell members are adjacent and cover a section of the front and sides of the user's leg; a pair of hinge pins disposed laterally on opposite sides of the knee joint attaching the said lower shell member and the said upper shell member together, such that said members may pivot about a primary pivot axis to allow bending of the knee joint. The knee brace further comprising at least two strap members attached to said shell members and extensible about the leg of the user.
A.) The knee brace according to claim 1, wherein an elasticized sleeve is used to secure the device to the user's leg.

2.) The knee brace of claim 1, wherein the distal section rotates on the axis of the connecting pins wherein its overlapping edge comes into contact with the outer surface of the proximal section, thereby preventing any further rotation of the device and effectively protecting against hyperextension of the user's leg and knee joint.

3.) The knee brace of claim 1, wherein the brace includes extended flanges on the side of the proximal member to conform to the upper calf area of the user's leg and stabilizes the brace below the knee joint and extended flanges on the side of the distal member to conform to the lower thigh area of the user's leg and stabilizes the brace above the knee joint to prevent lateral movement of the user's leg and knee joint.
